(19) United States
(12) Patent Application Publication
(10) Pub. No.: US 2025/0327741 A1
(43) Pub. Date: Oct. 23, 2025

(54) ALKENE DETECTION OF OZONE

(71) Applicant: Hach Company, Loveland, CO (US)

(72) Inventor: Chloe Morton Schroeder, Dacono, CO (US)

(21) Appl. No.: 18/639,371

(22) Filed: Apr. 18, 2024

(51) Int. Cl. G01N 21/31

(52) U.S. Cl. CPC G01N 21/31

(57) ABSTRACT

An embodiment provides a method for measuring ozone in a sample, including: introducing at least one alkene to a sample, wherein the alkene in the presence of an amount of ozone produces a carbonyl solution; adding 3-methyl-2-benzothiazolinonehydrazone hydrochloride (MBTH) to the carbonyl solution, and thereafter adding an oxidant to produce at least one imine and oxidized MBTH; producing a conjugated indicator from a reaction of the at least one imine and the oxidized MBTH; and measuring, using a colorimetric technique, an amount of ozone. Other aspects are described and claimed.

### BACKGROUND

This application relates generally to measuring ozone in aqueous or liquid samples, and, more particularly, to the measurement of ozone using an alkene and 3-methyl-2-benzothiazolinonehydrazone hydrochloride (MBTH).

Ensuring water quality is critical in a number of industries such as pharmaceuticals and other manufacturing fields. Additionally, ensuring water quality is critical to the health and well-being of humans, animals, and plants which are reliant on the water for survival. One element that is typically measured is ozone. Ozone may be controlled to make water safe for humans, animals, and aquatic life. Therefore, detecting the presence and concentration of ozone in water, food materials, or other liquid solutions is vital.

### BRIEF SUMMARY

In summary, one embodiment provides a method for measuring ozone in a sample, comprising: introducing at least one alkene to a sample, wherein the alkene in the presence of an amount of ozone produces a carbonyl solution; adding 3-methyl-2-benzothiazolinonehydrazone hydrochloride (MBTH) to the carbonyl solution, and thereafter adding an oxidant to produce at least one imine and oxidized MBTH; producing a conjugated indicator from a reaction of the at least one imine and the oxidized MBTH; and measuring, using a colorimetric technique, an amount of ozone.

Another embodiment provides a method for measuring ozone in a sample, comprising: introducing at least one alkene to a sample, wherein the alkene in the presence of an amount of an amount of ozone produces a carbonyl solution; adding 3-methyl-2-benzothiazolinonehydrazone hydrochloride (MBTH) to the carbonyl solution; and measuring, using a colorimetric technique, the amount of ozone.

A further embodiment provides a measurement device which measures ozone in a sample, comprising: an apparatus with a plurality of chambers for the measurement of ozone in a sample; a first chamber to introduce at least one alkene to the sample, wherein the alkene in the presence of ozone produces a carbonyl solution; a second chamber to add, 3-methyl-2-benzothiazolinonehydrazone hydrochloride (MBTH) to the carbonyl solution; and a third chamber to measure, using a colorimetric technique, the amount of ozone in the sample.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a reaction scheme example of an indicator for measurement of ozone.

FIG. 2 illustrates another reaction scheme example of an indicator for measurement of ozone.

FIG. 3 illustrates a further reaction scheme example of an indicator for measurement of ozone.

FIG. 4A illustrates example data of linearity using TEA allyl bromide for measurement of ozone.

FIG. 4B illustrates a reaction scheme example of TEA allyl bromide for measurement of ozone.

FIG. 5 illustrates example data of precision using TEA allyl bromide for measurement of ozone.

FIG. 6 illustrates example data of temperature effects using TEA allyl bromide for measurement of ozone.

FIG. 7 illustrates example data of linearity using 1,2-Di(4-pyridyl)ethylene for measurement of ozone

FIG. 8A illustrates example data of precision using 1,2-Di(4-pyridyl)ethylene for measurement of ozone.

FIG. 8B illustrates a reaction scheme example of 1,2-Di(4-pyridyl)ethylene for measurement of ozone.

FIG. 8C illustrates example data using 1,2-Di(4-pyridyl)ethylene for measurement of ozone.

FIG. 8D illustrates further example data using 1,2-Di(4-pyridyl)ethylene for measurement of ozone.

FIG. 9 illustrates example data of temperature effects using 1,2-Di(4-pyridyl)ethylene for measurement of ozone.

FIG. 10A illustrates example data of a dose response curve using 1,2-Di(4-pyridyl)ethylene for measurement of ozone.

FIG. 10B illustrates example data of a dose response curve using 1,2-Di(4-pyridyl)ethylene for measurement of ozone.

FIG. 11 illustrates an example flow diagram of a method for measuring ozone in a sample.

FIG. 12 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Conventional methods of ozone measurement in water may have some limitations. For example, ozone measurement may be used to determine the quality of water. High concentrations of ozone may be harmful to animals, humans, and/or plants. Accordingly, as another example, a user or entity may want the ozone in a body of water to be under a particular threshold, therefore, the user may measure the ozone to determine if the amount of ozone is under that threshold.

A standard for free and ozone measurement in water is Hach's AccuVac available from Hach Company, Loveland CO, USA (AccuVac is a registered trademark of Hach Company in the United States and other countries) which is a bleaching chemistry. These methods result in a bleaching of color in an amount proportional to the ozone concentration. The resulting color from the colorimetric reaction may be determined photometrically, for example, using a spectrophotometer. The amount of ozone may be determined by comparison to a similarly prepared blank vial. The absorbance of the sample reacted vial must be compared to the absorbance of the unreacted blank vial to determine the ozone concentration of the sample reacted vial.

However, the current analyte testing methods have limitations which are overcome by the methods and techniques as described in more detail herein. One limitation of the current techniques is that they use a bleaching chemistry not favorable to some users and measurement systems. Additionally, the traditional colorimetric methods require the preparation of a separate “blank” vial. The extra step of preparing a blank vial can introduce error to the measurement based upon individual human techniques in preparing the blank. Also, since the traditional colorimetric technique involves the bleaching of a dye, the time for preparation and time a measurement is taken, can introduce variability in the sample reading. Additionally, because the techniques include bleaching of a dye, difficulty may arise because there may not be the same amount of starting colorimetric dye in both the blank and sample vial, thereby introducing error into the determination of the amount of analyte found in the sample. This error may result in a false positive or false negative result.

Accordingly, an embodiment provides a system and method for measuring ozone in a sample. The sample may be drawn from a volume of liquid such as a holding tank, water source, food material source, beverage source, or the like. In an embodiment, the method may detect ozone in concentrations in about the range of 20 parts per billion (ppb)-2.0 parts per million (ppm). In an embodiment, the method may use a colorimetric measurement method. The indicator to give a colorimetric signal may be derived by a reaction with at least one alkene. In an embodiment, the color or colorimetric intensity may be correlated to the detection or concentration of ozone in a sample. A buffer, such as phosphate buffer, may be added. The pH may be adjusted to about pH 7.0 or lower. In an embodiment, treatment of a water sample containing ozone with a water-soluble alkene affords two carbonyls. The carbonyl may be at least one aldehyde and/or at least one ketone. These carbonyls undergo dehydration with addition of 3-methyl-2-benzothiazolinonehydrazone hydrochloride (MBTH) to afford the condensed imines. Depending on the alkene of choice, these imines may also be quantifiable by a colorimetric method. Otherwise, oxidation of excess MBTH promotes further condensation to a blue product that can be analyzed at about 655 nm.

The illustrated example embodiments will be best understood by reference to the figures. The following description is intended only by way of example, and simply illustrates certain example embodiments.

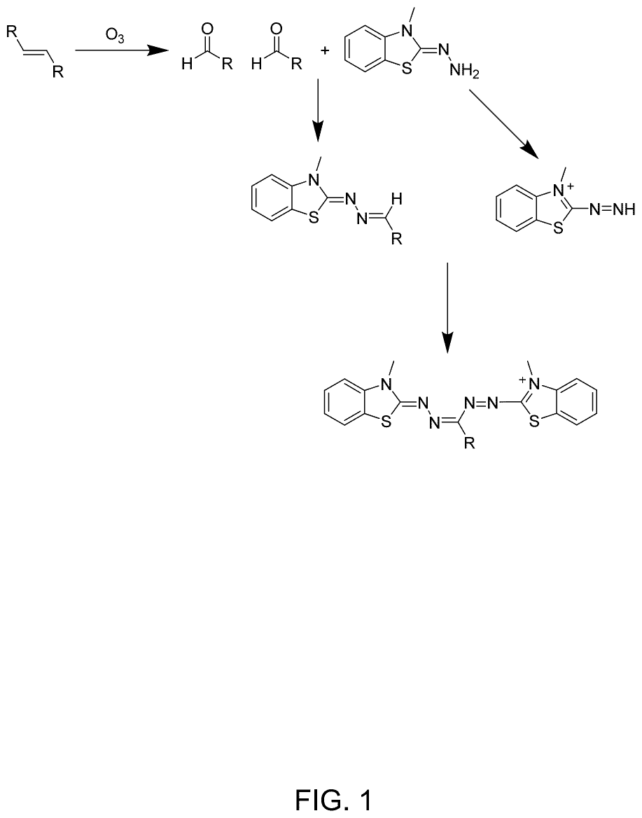

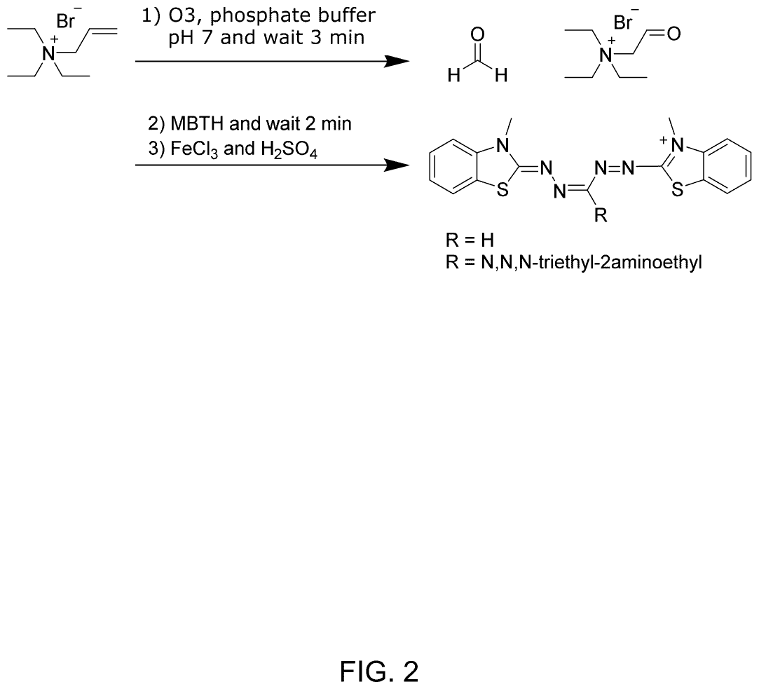

Referring to FIG. 1, in an embodiment, an example method for detection of ozone in a sample is illustrated. In an embodiment, an alkene of choice may be treated with or exposed to ozone to create two aldehydes. In an embodiment, the alkene may be a mono-substituted alkene or a 1,2-disubstituted alkene in order to form 2 aldehydes. As examples, a trisubstituted akene will produce one aldehyde and 1 ketone, and a tetrasubstituted alkene will only afford ketones and will not work for the scheme of FIG. 2. A tetrasubstituted alkene (affording ketones) may work in the scheme of FIG. 1 if there is no reaction with FeCl₃ and an acid. Treatment of the aldehyde products with 3-methyl-2-benzothiazolinonehydrazone hydrochloride (MBTH) yields two imines. After the imines have been allowed to form, excess MBTH may be oxidized by ferric chloride in the presence of sulfuric acid to yield oxidized MBTH. The oxidized MBTH reacts with imine to yield a conjugated indicator. Other acids, oxidizing agents, or oxidants may be used.

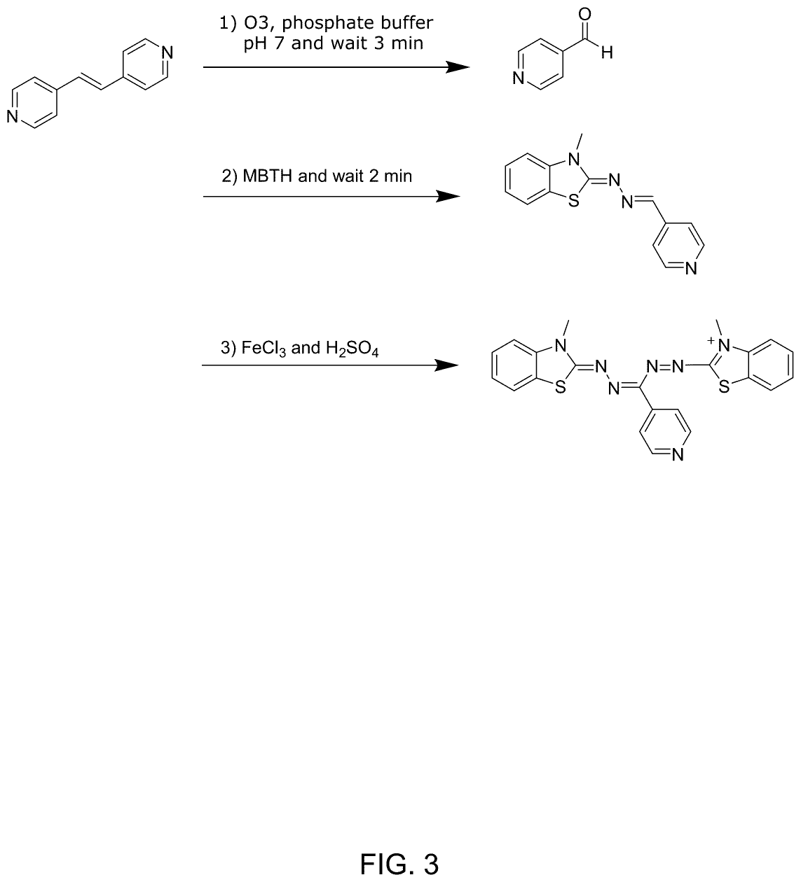

In an embodiment, the alkene may be a solid and water soluble for application of ozone detection in the water industry or an aqueous sample. Additionally, if the two R groups of a 1,2-disubstituted alkene are the same, one equivalent of ozone affords two equivalents of the same conjugated indicator, increasing the intensity of the signal by visible detection. Different aldehydes were tested with MBTH followed by oxidation with FeCl₃ and H₂SO₄ to determine which functional groups may limit testing. Other acids may be used such as sulfamic acid or another strong acid. H₂SO₄ is an exemplar acid. The reactions disclosed herein work on a variety of alkenes; however, incompatible functional groups may include sulfonic acids, carboxylic acids, and some phenols. A distinct color change of some alkenes after reaction with ozone followed by MBTH allow for a quantification of ozone concentration in water or an aqueous sample without addition of FeCl₃ and acid. In an embodiment, different experimental conditions may be used. Some example conditions are illustrated in FIG. 2 and FIG. 3.

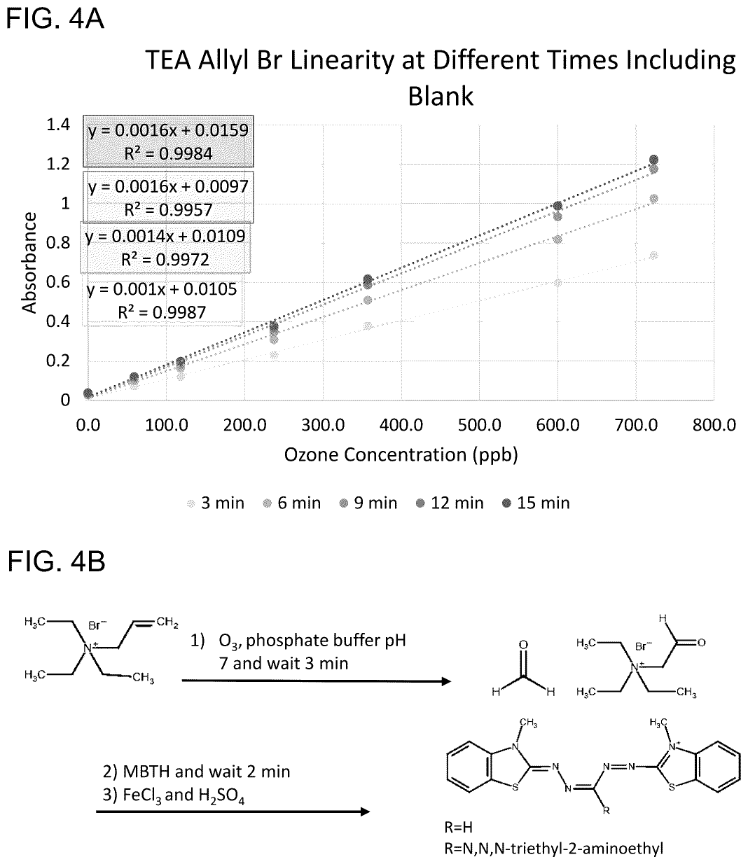

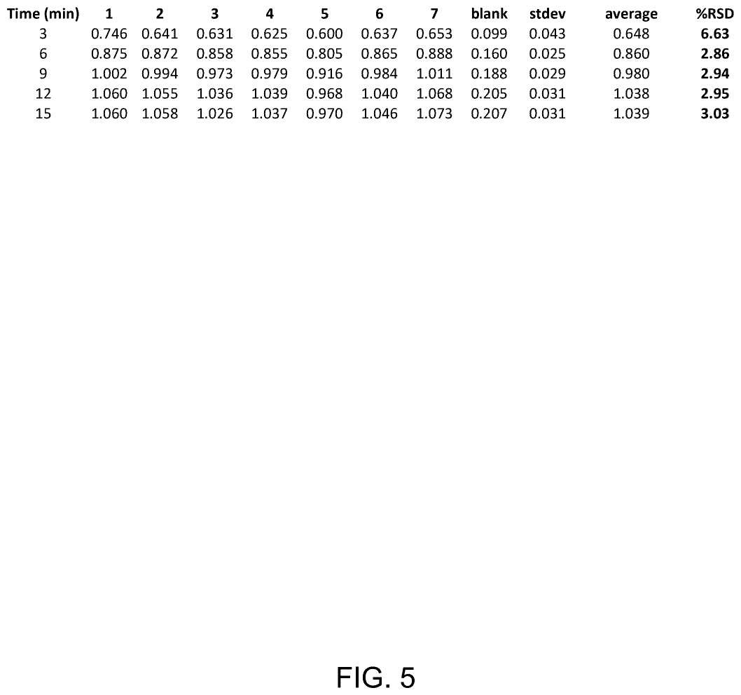

Referring to FIG. 4A, in an embodiment, a linear dose response curve of TEA allyl bromide after treatment of ozone, MBTH, then FeCl₃ and H₂SO₄ or conjugated indicator over time is illustrated. For this example, T=0 is on addition of the aqueous mixture of FeCl₃ and H₂SO₄. As the reaction goes to completion, the slope of the linear curve increases. Linearity is maintained throughout the reaction. Therefore, the reaction could be monitored at a chosen timepoint greater than 3 minutes after addition of FeCl₃. A colorimetric change was measured at 655 nm. Referring to FIG. 4B, in an embodiment, a reaction and experimental scheme for these example data are illustrated. Referring to FIG. 5, the precision of the method is illustrated. By the time point at 6 min after adding the FeCl₃, the % RSD (relative standard deviation) was consistently ˜3% for this example. The alkene and MBTH causes a color change in the sample upon detection of ozone. The color may depend on the alkene used for the reaction. As examples, the color may be blue, yellow, or green (a combination of blue and yellow).

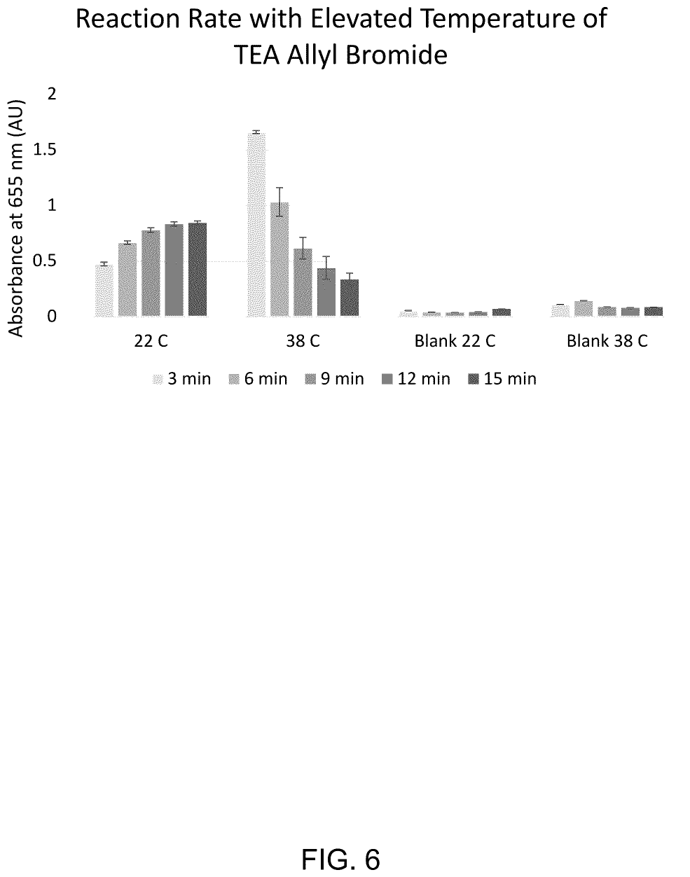

Referring to FIG. 6, in an embodiment, data for temperature elevation to drive the formation of the conjugated indicator is illustrated. For this example, the reaction was complete between 0 and 6 minutes when heated to 38 degrees Celsius compared to 12 minutes at 22 degrees Celsius. The conjugated indicator formed from reaction of alkene (TEA allyl bromide) may decompose, and decomposition may occur within 30 minutes at room temperature, which explains a decrease in absorbance in the 38 degrees Celsius samples after 6 minutes. Optimization of temperature and time may be made to quantitate ozone concentration by the use of a given alkene. Samples at 22 and 38 degrees Celsius were run in triplicate with error bars based upon a standard deviation.

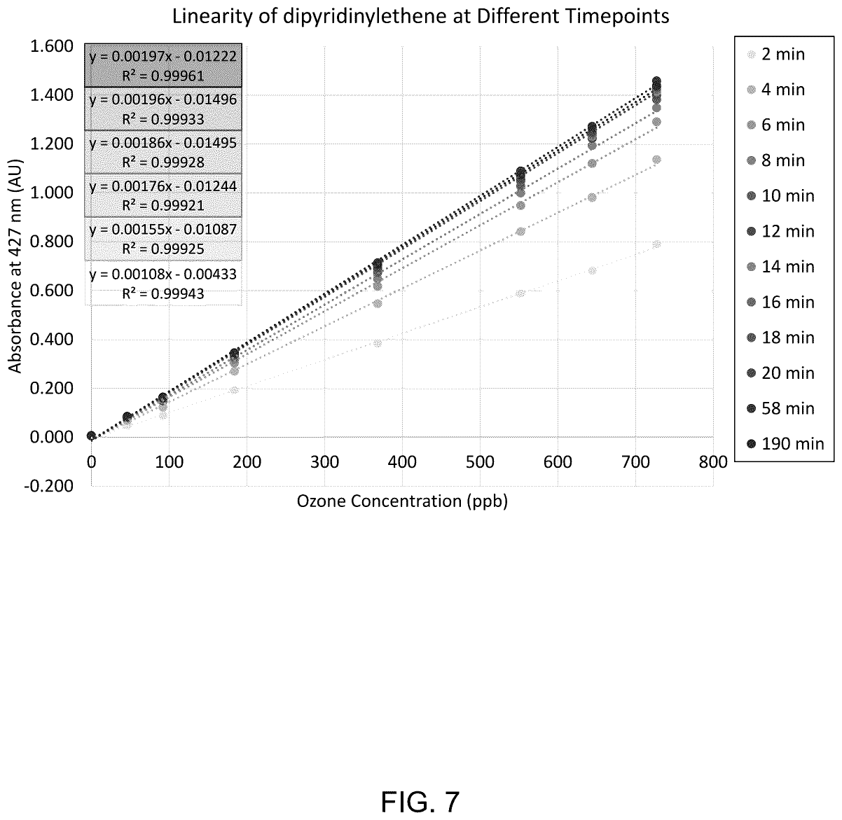

Referring to FIG. 7, in an embodiment, sample data showing linearity of treatment of an alkene (1,2-Di(4-pyridyl)ethylene) with ozone followed by MBTH is illustrated. In this example, T=0 is on addition of the aqueous mixture of MBTH. As the reaction goes to completion, the slope of the linear curve increases. Linearity is maintained throughout the reaction. Therefore, the reaction may be monitored at a chosen timepoint greater than 2 minutes after addition of MBTH. Unlike the instability of indicator illustrated above (TEA allyl bromide), this imine was stable over 190 minutes, providing more flexibility in increasing the rate of reaction by an increase in temperature. A colorimetric change was measure at 427 nm.

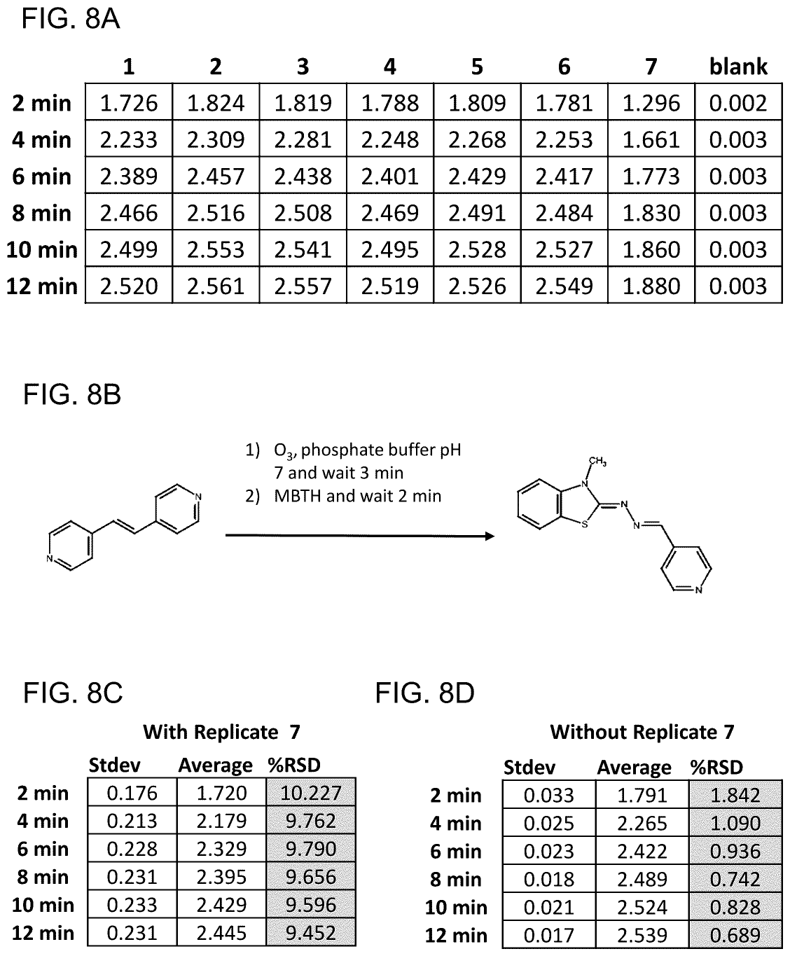

Referring to FIG. 8, in an embodiment, precision data performed on this method are illustrated (1,2-Di(4-pyridyl)ethylene with ozone followed by MBTH to afford an imine). The raw data is shown in FIG. 8A. Referring to FIG. 8B, in an embodiment, a reaction and experimental scheme for these example data are illustrated. Replicate 7 was an outlier and presented are the inclusion and exclusion of replicate 7 (FIG. 8C and FIG. 8D, respectively). Including sample 7, the % RSD was ˜10%. Excluding replicate 7, the % RSD was ˜1%.

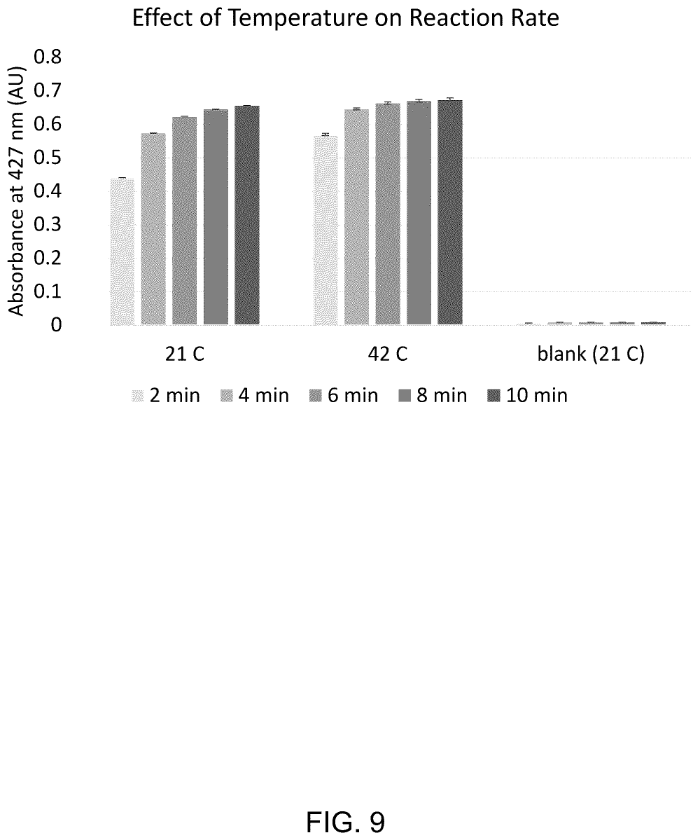

Referring to FIG. 9, in an embodiment, the reaction rate of formation of imine was found to increase with an increase in temperature as illustrated. For example, when heated to 42 degrees Celsius, the maximum absorbance was reached at ˜6 min, about half the time necessary compared to 21 degrees Celsius (10 min). The ideal temperature and time may be determined based on experimental conditions to quantify ozone concentration.

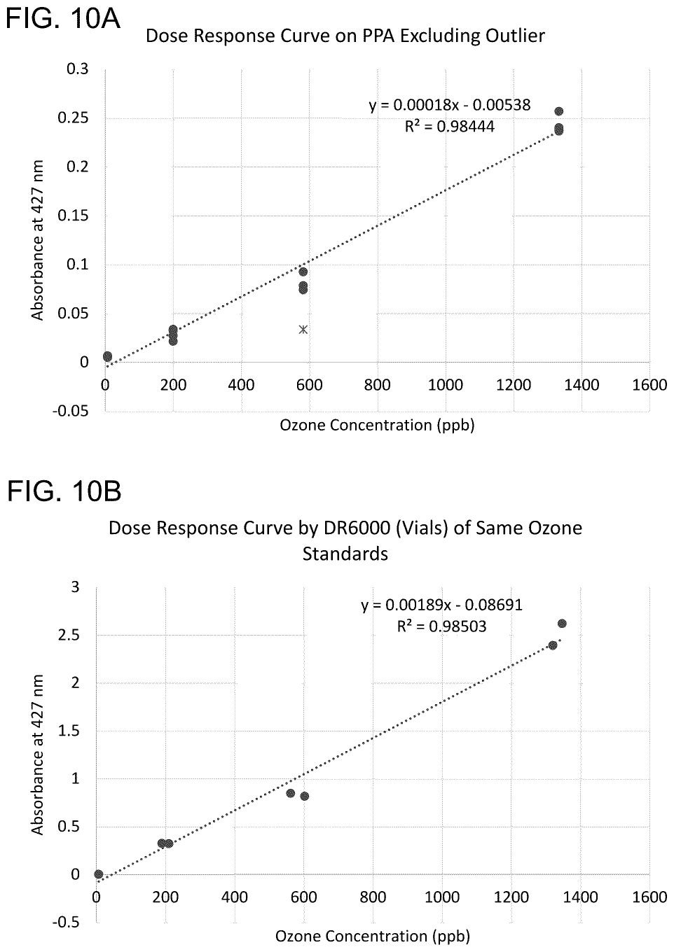

Referring to FIG. 10A, in an embodiment, an example dose response curve is illustrated using a portable parallel analyzer (PPA). Referring to FIG. 10B, a dose response curve using a DR6000 instrument (available from Hach Company, Loveland CO, USA). Both methods in FIG. 10A and FIG. 10B utilized the same standard ozone solutions whose concentrations were determined by the DPD method.

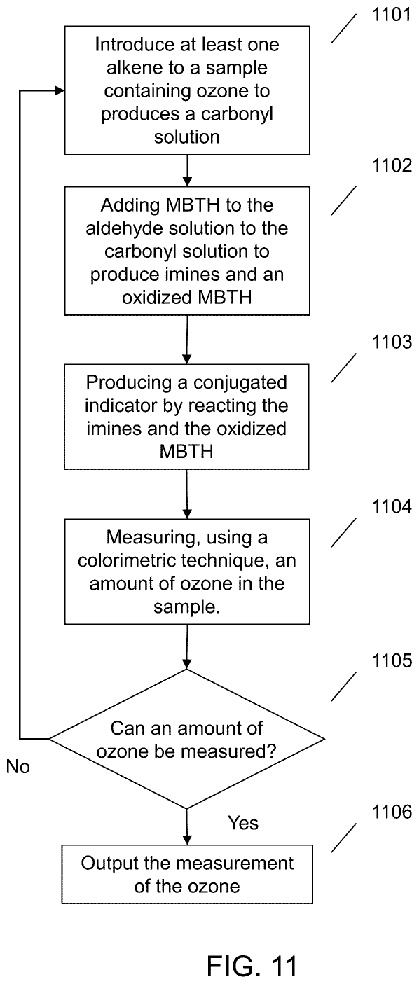

Referring to FIG. 11 at 1101, in an embodiment, an example method of ozone detection is illustrated. For example, at least one alkene may be introduced into a sample. The addition of at least one alkene may yield carbonyls, or a carbonyl solution. As an example, a symmetrical alkene may yield two identical carbonyls. The sample may contain ozone or an amount of ozone. The solution may be a sample which may include a sample from a natural body of water, a holding tank, a processing tank, a pipe, a water system, a volume of liquid for food preparation, or the like. The solution may be in a continuous flow, a standing volume of liquid, or any combination thereof. In one embodiment, the sample may be introduced to the reagents for the method, for example, a test chamber of the measurement device. Introduction of the sample into the measurement device may include placing or introducing the sample into a test chamber manually by a user or using a mechanical means, for example, gravity flow, a pump, pressure, fluid flow, or the like. For example, a water sample for ozone testing may be introduced to a measurement or test chamber using a pump. In an embodiment, valves or the like may control the influx and efflux of the solution into or out of the one or more chambers, if present.

Additionally, or alternatively, the measurement device may be present or introduced in a volume of the sample. The measurement device is then exposed to the volume of sample where it can perform measurements. The system may be a flow-through system in which a solution and/or reagents are automatically mixed and measured. Once the sample is in contact with the measurement system, the system may measure the ozone of the sample or a change in color of the sample, as discussed in further detail herein. In an embodiment, the measurement device may include one or more chambers in which the one or more method steps may be performed. In an embodiment, the pH of the solution may be controlled or buffered. The buffer may be a phosphate buffer.

At 1102, in an embodiment, MBTH may be added to the sample or reaction. The addition of MBTH yields imines. After the imines have been allowed to form, excess MBTH may be oxidized by ferric chloride in the presence of sulfuric acid, sulfamic acid, or the like to afford the oxidized MBTH. At 1103, the oxidized MBTH reacts with imine to yield the conjugated indicator. In an embodiment, conjugated indicator or indicator in the presence of ozone may “turn-on” the colorimetric properties for measurement of ozone in a sample.

At 1104, in an embodiment, the system and method may measure ozone, ozone concentration, or an amount of zone in a sample. In an embodiment, the presence of ozone in a sample may cause an increase in colorimetric intensity of the indicator. Examples of this increase in colorimetric intensity and dose response curves for an indicator are illustrated herein. Experimental conditions may include measurement of ozone at room temperature or heating the sample to up to 55 degrees Celsius. The sample may be stirred. Steps of the method may be performed in series, or simultaneously. These conditions are exemplary and may be altered based upon conditions.

The measured color may be correlated to a concentration or amount of ozone in the sample or water. The sample may be diluted with deionized water depending on the ozone concentration. Measurements may be made using the Hach SL1000, Hach DR6000 (available from Hach Company, Loveland CO, USA) instrument, or an equivalent spectrometer instrument. In an embodiment, an excitation wavelength may be between 420 and 660 nanometers (nm).

Therefore, the colorimetric intensity, of a solution or sample containing ozone may be correlated to the intensity of a change in the intensity in the sample or aqueous solution. Colorimetric curves may be generated for a range of concentrations, for different indicators from different alkenes, for any different condition that may affect colorimetric values (e.g., temperature, sample content, turbidity, viscosity, measurement apparatus, aqueous sample chamber, etc.), or the like.

Alternatively, or additionally, ozone measurement may be at periodic intervals set by the user or preprogrammed frequencies in the device. Measurement by a device allows for real time data with very little human involvement in the measurement process. Cleaning of the measurement chamber may be required at an unspecified time interval. A programmed calibration curve may be entered into the device.

A chamber, vessel, cell, chamber, or the like may contain a sample, at least one indicator, and associated reagents such as buffers, additives, MBTH, alkene(s), or the like. A device may contain one or more bottles of reagents which contain necessary reagents. The reagents contained in the one or more bottles may be pump fed or gravity fed. The flow of the reagents may be metered to ensure proper volume delivery to the measurement cell. The sample may be fed through a pressured inlet, a vessel, or the like. The sample may be introduced into the measurement chamber by a pump or gravity fed. The sampling device may be in series or parallel to an aqueous flow. The device may have a system to ensure proper mixing and/or temperature of the aqueous sample and related reagents.

At 1106, in an embodiment, the colorimetric intensity or ozone concentration may be an output upon a device in the form of a display, printing, storage, audio, haptic feedback, or the like. Alternatively, or additionally, the output may be sent to another device through wired, wireless, fiber optic, Bluetooth®, near field communication, or the like. An embodiment may use an alarm to warn of a measurement or concentration outside acceptable levels. An embodiment may use a system to shut down water output or shunt water from sources with unacceptable levels of an analyte. For example, an analyte measuring device may use a relay coupled to an electrically actuated valve, or the like.

At 1105, in an embodiment, if a concentration of ozone cannot be determined, the system may continue to measure ozone and/or changes in colorimetric intensity. Additionally, or alternatively, the system may output an alarm, log an event, or the like.

If a concentration of ozone can be determined, the system may provide a measurement of ozone concentration at 1106. The system may connect to a communication network. The system may alert a user or a network. This alert may occur whether an ozone measurement is determined or not. An alert may be in a form of audio, visual, data, storing the data to a memory device, sending the output through a connected or wireless system, printing the output or the like. The system may log information such as the measurement location, a corrective action, geographical location, time, date, number of measurement cycles, or the like. The alert or log may be automated, meaning the system may automatically output whether a correction was required or not. The system may also have associated alarms, limits, or predetermined thresholds. For example, if an ozone concentration reaches a threshold. Alarms or logs may be analyzed in real-time, stored for later use, or any combination thereof.

Alternatively, the method and measurement described herein may be performed with a chemistry on chip product. For example, using the Chemkeys® available from Hach Company, Loveland CO, USA (ChemKey® is a registered trademark of Hach Company in the United States and other countries) and using the Hach SL1000 (available from Hach Company, Loveland CO, USA). For example, the chemistry and reagents necessary for the measurement of ozone may be preloaded or present upon a Portable Parallel Analyzer (PPA) from Hach Company, Loveland CO, USA (Portable Parallel Analyzer is a registered trademark of Hach Company in the United States and other countries) or chip. For example, a sample of water containing ozone may be loaded or presented to the ChemKey®. The ChemKey® may be preloaded with reagents. The reagents may be placed in a proper chamber and the chambers in a proper order such that the sample may travel through the different chemistries, reagents, or steps of the method. The analyzer may control a temperature of the reaction. The associated measurement device may then take a colorimetric measurement at the end of the process. This presents a streamlined, less error prone measurement method to an end user. As an example, a 6-slot Chemkey may use the 3 first slots for alkene introduction, the fourth for MBTH, fifth for FeCl₃, and the sixth for mixing and/or measurement. An additional methodology, such as a powder pillow may be used in which reagents are prepackaged in an amount for introduction into a reaction vessel.

The various embodiments described herein thus represent a technical improvement to conventional ozone measurement techniques. Using the techniques as described herein, an embodiment may use an indicator as synthesized herein to measure ozone in solution. This is in contrast to methodology with limitations mentioned above. Such techniques provide a faster and more accurate method for measuring ozone in an aqueous or liquid solution. The various embodiments described herein thus represent a technical improvement to precise ozone measurement in a sample. Using the techniques as described herein, an embodiment may use a method and device to measure ozone concentration. This is in contrast to conventional methods with limitations mentioned above.

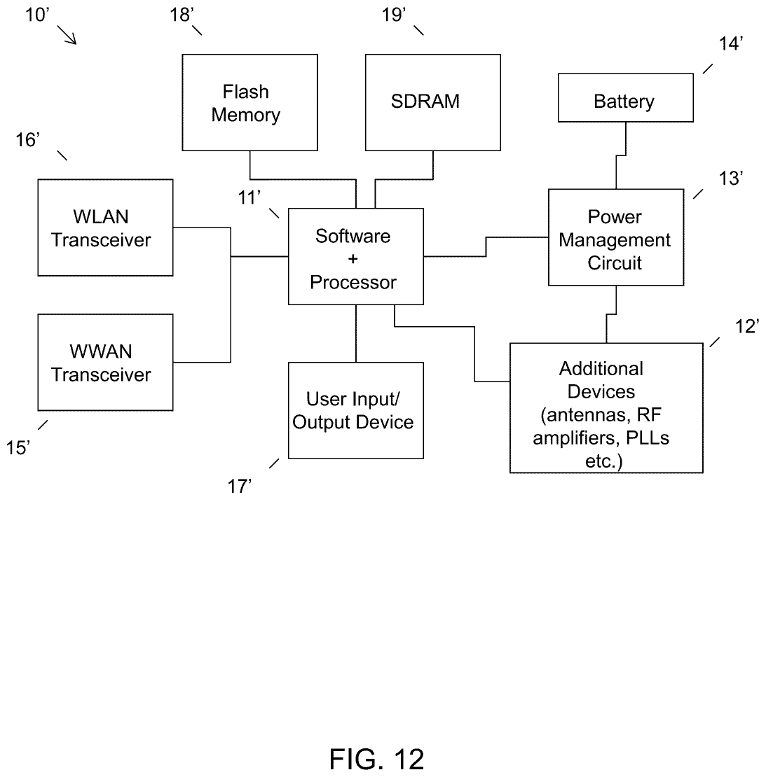

While various other circuits, circuitry or components may be utilized in information handling devices, with regard to an instrument for ozone measurement in according to any one of the various embodiments described herein, an example is illustrated in FIG. 12. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment to perform ozone measurement of a sample.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a handheld measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for measuring ozone in a sample, comprising:
introducing at least one alkene to a sample, wherein the alkene in the presence of an amount of ozone produces a carbonyl solution;
adding 3-methyl-2-benzothiazolinonehydrazone hydrochloride (MBTH) to the carbonyl solution, and thereafter adding an oxidant to produce at least one imine and oxidized MBTH;
producing a conjugated indicator from a reaction of the at least one imine and the oxidized MBTH; and
measuring, using a colorimetric technique, an amount of ozone.

2. The method of claim 1, wherein the at least one alkene is a symmetrical amine.

3. The method of claim 1, further comprising adjusting the sample to a pH of about 7.0 or less.

4. The method of claim 1, further comprising heating the sample.

5. The method of claim 1, further comprising adding a phosphate buffer with the at least one alkene.

6. The method of claim 1, wherein an amount of ozone in the sample is in the range of 20 parts per billion-2.0 parts per million.

7. The method of claim 1, wherein the colorimetric intensity is correlated to a concentration of the ozone in the sample.

8. The method of claim 1, wherein the colorimetric measuring uses a wavelength between 420 and 660 nm.

9. The method of claim 1, further comprising a portable parallel analyzer.

10. The method of claim 1, wherein the oxidant comprises FeCl₃ and an acid.

11. A method for measuring ozone in a sample, comprising:
introducing at least one alkene to a sample, wherein the alkene in the presence of an amount of an amount of ozone produces a carbonyl solution;
adding 3-methyl-2-benzothiazolinonehydrazone hydrochloride (MBTH) to the carbonyl solution; and
measuring, using a colorimetric technique, the amount of ozone.

12. The method of claim 11, wherein the at least one alkene is a symmetrical amine.

13. The method of claim 11, further comprising adjusting the sample to a pH of about 7.0 or less.

14. The method of claim 11, further comprising heating the sample.

15. The method of claim 11, further comprising adding a phosphate buffer with the at least one alkene.

16. The method of claim 11, wherein an amount of ozone in the sample is in the range of 20 parts per billion-2.0 parts per million.

17. The method of claim 11, wherein the colorimetric intensity is correlated to a concentration of the ozone in the sample.

18. The method of claim 11, wherein the colorimetric measuring uses a wavelength between 420 and 660 nm.

19. The method of claim 11, further comprising a portable parallel analyzer.

20. A measurement device which measures ozone in a sample, comprising:
an apparatus with a plurality of chambers for the measurement of ozone in a sample;
a first chamber to introduce at least one alkene to the sample, wherein the alkene in the presence of ozone produces a carbonyl solution;
a second chamber to add, 3-methyl-2-benzothiazolinonehydrazone hydrochloride (MBTH) to the carbonyl solution; and
a third chamber to measure, using a colorimetric technique, the amount of ozone in the sample.
